(19) United States
(12) Patent Application Publication
(10) Pub. No.: US 2025/0387320 A1
(43) Pub. Date: Dec. 25, 2025

(54) ACIDIC, PRESERVATIVE-FREE SKIN-BARRIER REPAIR COMPOSITION

(71) Applicant: Vinoja Wichweswaran, Brampton (CA)

(72) Inventor: Vinoja Wichweswaran, Brampton (CA)

(21) Appl. No.: 19/243,811

(22) Filed: Jun. 20, 2025

Related U.S. Application Data
(60) Provisional application No. 63/662,002, filed on Jun. 20, 2024.

(51) Int. Cl. A61K 9/00; A61K 36/185; A61K 36/899; A61K 47/14

(52) U.S. Cl. CPC A61K 9/0014; A61K 36/185; A61K 36/899; A61K 47/14

(57) ABSTRACT

The present disclosure relates to an acidic, preservative-free skin-barrier repair composition for application to human skin, the composition comprising at least one emollient; at least one physiologically compatible acid suitable for topical application to allow for the progressive re-acidification of the skin after barrier disruption; at least one naturally-sourced or non-harming anti-inflammatory agent; and optionally a vasodilator.

### CROSS-REFERENCE TO RELATED APPLICATION

The present disclosure claims the benefit of U.S. Provisional Application No. 63/662,002, filed on Jun. 20, 2024 which is incorporated herein by reference.

### FIELD

The present disclosure relates to a skin-barrier repair composition and, more particularly, to a topical, acidic, preservative-free skin-barrier repair composition.

### BACKGROUND

Barrier disruption can be triggered by a wide array of intrinsic and extrinsic factors. Common causes include excessive cleansing or use of harsh surfactants, over-exfoliation with physical or chemical agents, and frequent exposure to hot water, all of which can strip the skin of its natural lipids and acids. Environmental stressors such as low humidity, cold weather, wind, and pollution can further compromise the barrier by increasing transepidermal water loss and oxidative stress. Additionally, the use of certain topical medications—such as retinoids, corticosteroids, or benzoyl peroxide—can impair barrier function, especially when used inappropriately or for prolonged periods. Underlying medical conditions, including atopic dermatitis, psoriasis, and ichthyosis, are associated with genetic or acquired defects in barrier proteins and lipids, predisposing individuals to chronic barrier dysfunction. Lifestyle factors such as poor nutrition, psychological stress, and lack of sleep may also negatively impact the skin's ability to repair itself. Finally, age-related changes, hormonal fluctuations, and immune dysregulation can all contribute to a weakened barrier, making the skin more susceptible to irritation, infection, and inflammation. Barrier disruption of the skin is a prevalent condition, typically occurring at least once in the lifetime of the average individual. Barrier disruption looks and feels like dryness, redness, flaking, itchiness, irritation and sometimes infection. Those with a genetic predisposition to barrier disruption, like those that have atopic dermatitis, (likely fueled by a genetic deficiency) will experience frequent and prolonged barrier disruption. Nonetheless, even individuals with otherwise healthy skin may undergo extended periods of barrier impairment as a result of the factors discussed above, frequently leading to or exacerbated by heightened inflammatory responses.

Thus, barrier disruption leads to at least a low-level of skin inflammation. Prompt and effective repair of the skin barrier is crucial as, once the barrier is compromised, a self-perpetuating cycle of inflammation and further barrier disruption can quickly ensue (see FIG. 1). The applicant has identified the importance of early intervention, through acidification and healing of the various components and layers of the skin barrier, in maintaining skin barrier homeostasis which has not previously been identified in any of the prior art. In the literature it is know that when the skin's protective layer is disrupted, irritants, allergens, and pathogens can penetrate more easily, triggering an immune response that leads to further inflammation and damage to the skin barrier, but a comprehensive solution does not exist. This inflammatory cascade not only delays healing but can also worsen the initial injury, making the skin more susceptible to chronic conditions and recurrent flare-ups. If the barrier is not rapidly restored, the ongoing inflammation can further impair the skin's ability to repair itself, resulting in prolonged discomfort, increased sensitivity, and a heightened risk of developing persistent dermatological issues. Therefore, interventions that accelerate barrier recovery are essential to break this cycle and restore skin health efficiently. However, even products specifically designed to repair major or ongoing skin irritation do not take into consideration the importance of a comprehensive approach or the importance of re-acidifying the skin barrier.

Despite its critical defensive role, the skin barrier is inherently fragile and susceptible to a variety of insults. As mentioned, everyday exposures such as harsh cleansers, environmental pollutants, UV radiation, and even frequent washing can compromise the delicate balance of the barrier's multiple layers. The physical layer, composed of corneocytes and intercellular lipids, can be eroded by mechanical abrasion or chemical irritants. The chemical layer, including the acid mantle, is easily disrupted by alkaline products or excessive sweating, among other factors. The microbiome, a community of beneficial microorganisms, can be thrown off balance by antibiotics or antimicrobial agents, while the immunologic layer may be overwhelmed by allergens or pathogens. Each of these assaults, are not an exhaustive list of factors, but can nonetheless independently or synergistically weaken the barrier, setting the stage for the cycle of inflammation and impaired repair described above.

Thus, the integrity of the skin barrier is maintained by a combination of physical, chemical, microbiome, and immunologic layers. Disruption of any of these layers can result in increased transepidermal water loss (TEWL), elevated skin pH, dysbiosis of the skin microbiome, and heightened inflammation, each or all of which demonstrate a disruption of the skin barrier and are manifested clinically as dryness, erythema, pruritus, and increased susceptibility to infection.

A critical, yet often overlooked, aspect of skin barrier health is the maintenance of an acidic surface pH (the “acid mantle”), typically between 4.0 and 6.0. This acidity is essential for: regulating key enzymes involved in lipid processing and barrier formation (e.g., β-glucocerebrosidase, acid sphingomyelinase); supporting the growth of beneficial commensal flora (e.g., Staphylococcus epidermidis) while suppressing pathogenic microbes (e.g., Staphylococcus aureus); promoting barrier repair and reducing inflammation; optimising the activity of proteases and other enzymes involved in desquamation and cornification. Disruption of the acid mantle, common when using alkaline, or irritating skincare products or caused through environmental insults, impairs these processes, leading to barrier dysfunction and inflammation.

Existing barrier repair products do not focus on importance of the re-acidification of the skin, while also maintaining the integrity of the other layers of the skin barrier.

Steroidal compounds (e.g., hydrocortisone, betamethasone, triamcinolone), although not intended for barrier repair, are widely used to suppress inflammatory responses in the skin by downregulating pro-inflammatory cytokines and mediators. The effect can be rapid in reducing redness, swelling, and irritation. However, while steroids can sometimes sufficiently tame inflammation to allow for the skin barrier to recover from barrier disruption, their function is not to heal the skin barrier but to prevent inflammation from escalating. Unfortunately, while they may provide some relief, they can also cause skin damage (including skin thinning) and therefore hinder rather than aid in barrier repair. Other pharmaceutical options, with similar objectives, like immunomodulators and immunosuppressants, similarly can create more harm to the skin barrier. Like steroids, they do not typically help rebuild or maintain a healthy stratum corneum; in fact, extended use can thin the epidermis and impair natural barrier function, potentially exacerbating dryness and sensitivity over time. Thus, while steroids and other similar pharmaceuticals can reduce inflammation in the short term, they ultimately do not restore the lipids and structural proteins crucial for sustained barrier integrity.

Further, there are many barrier repair products on the market, but none address the elements of the skin barrier adequately. The health of the skin barrier relies on several key factors: maintaining the skin's natural acidity, protecting the delicate balance of the skin's microbiome, and retaining moisture through natural moisturizing factor (NMF). In addition, once the skin barrier is disrupted, as mentioned, there will also be some skin inflammation that will prevent the skin from healing. Therefore, to address the immunologic layer of the skin, inflammation needs to be downregulated through topical anti-inflammatories.

Most products on the market address the moisturization component of skin healing and include anti-inflammatory ingredients (such as colloidal oatmeal) to help with the soothing of inflammation. The problem with this approach is that those cosmetic formulations may include preservatives or broad-spectrum antibacterials and/or not be adequately acidic.

Hence, commercially available barrier repair products fail to address the various aspects of skin barrier health. Commercially available pH balanced moisturizers, while acidic in nature, do not take into consideration the importance of anti-inflammatories or microbiome support in the repair of the skin barrier. Furthermore, the challenge of formulating a preservative-free, acidic product that provides for the re-acidification of the skin has not been adequately addressed in the art. It is important to note that, in this context, the term ‘preservative-free’ refers specifically to the absence of typical preservatives commonly used in water-based formulations. These ingredients, while effective at preventing microbial growth, can sometimes disrupt the skin barrier or microbiome, and their exclusion presents unique formulation challenges, particularly in maintaining product safety and stability without compromising skin health.

Modulating between the percentage of acids vs anti-inflammatories in a skincare formulation can help restore skin barrier health. To clarify further, initial barrier disruption, often characterized by mild dryness and redness, can often be healed through the use of a good moisturizer (although the results are not always consistent). However, quick and effective healing is aided by the acidification of the skin upon initial barrier disruption. Disrupting the skin's physical barrier experimentally causes an increase in pH, which takes several hours to return to normal. Many inflammatory skin conditions and diseases affecting the epidermis are associated with a compromised barrier and elevated skin pH.

However, the pH of the stratum corneum exhibits a gradient, being more acidic at the outermost surface and progressively approaching neutral values (approximately pH 7.0 to 7.4) in the underlying viable layers. This increase in average pH with depth is attributed to a reduction in the relative proportion of acidic microdomains compared to neutral regions within the stratum corneum. Therefore, the modulating nature of this composition can benefit a variety of skin conditions. Skin, with mild barrier disruption, can benefit from formulations that include slightly higher acidity combined with anti-inflammatory ingredients, as the acidic environment helps restore barrier function. However, when the skin barrier is severely compromised, a formulation with higher concentrations of anti-inflammatory agents and lower levels of acids is more appropriate, since prolonged barrier damage is associated with increased inflammation and a higher sensitivity to acidic components, as deeper layers of the skin may be exposed.

### SUMMARY

This section provides a general summary of the present disclosure and is not a comprehensive disclosure of its full scope or all of its features and advantages.

A critical, yet often overlooked, aspect of skin barrier health is the maintenance of an acidic surface pH (the “acid mantle”), typically between 4.0 and 6.0. This acidity is essential for: regulating key enzymes involved in lipid processing and barrier formation (e.g., β-glucocerebrosidase, acid sphingomyelinase); supporting the growth of beneficial commensal flora (e.g., Staphylococcus epidermidis) while suppressing pathogenic microbes (e.g., Staphylococcus aureus); promoting barrier repair and reducing inflammation; optimizing the activity of proteases and other enzymes involved in desquamation and cornification. Disruption of the acid mantle, often through the use of alkaline skincare products or environmental insults, impairs these processes, leading to barrier dysfunction and inflammation.

Therefore, an object of embodiments of the present disclosure is to provide a topical composition specifically designed for skin barrier repair through acidification, thereby helping to restore the skin's natural acidity and supporting overall skin health. This is achieved through a controlled content of one or more physiologically compatible acids, so as to improve surface pH and bring the skin's pH to its normally acidic state, thereby supporting the skin's acid mantle and barrier function. Physiologically compatible acid means any acid that is safe for topical application.

A further object of embodiments of the present disclosure is to provide a preservative-free formulation that avoids the use of broad-spectrum anti-bacterials or preservatives, which are known to disrupt the delicate balance of the skin microbiome and may contribute to irritation or sensitization, particularly in individuals with compromised skin barriers. By eliminating these potentially disruptive agents, the composition is designed to be especially suitable for sensitive, reactive, or allergy-prone skin types. This approach supports the skin's natural defense mechanisms and fosters an environment conducive to barrier repair and long-term skin health.

A further object of embodiments of the present disclosure is to incorporate anti-inflammatory agents that are naturally derived or otherwise non-harming or non-disruptive to the skin's delicate barrier. These agents are carefully selected to provide effective soothing and calming benefits without the adverse effects commonly associated with conventional steroidal or harsh anti-inflammatories, such as skin thinning or barrier compromise. By utilizing botanically sourced extracts, bioactive lipids, or other non-harming anti-inflammatory compounds, the composition aims to reduce redness, irritation, and discomfort, thereby supporting the skin's natural healing processes. This strategy ensures that individuals with sensitive, inflamed, or compromised skin can benefit from targeted inflammation control to promote barrier repair and resilience.

A further object of embodiments of the present disclosure is to provide a means for modulating the ratio between the percentage of physiologically compatible acids and anti-inflammatory agents within the composition. This modulation allows for tailored formulations that can be optimized for varying degrees of skin sensitivity, inflammation, or barrier impairment, as described earlier. By adjusting the relative concentrations of these key components, the composition can be customized to deliver the desired balance of skin acidification and inflammation control, thereby enhancing its efficacy for a broad spectrum of skin types and conditions.

Accordingly, it is an aspect of the present disclosure to provide a topical skin-barrier repair composition formulated to restore the skin's natural acidity, support a healthy and diverse microbiome, provide effective moisturization, reduce inflammation and restore the skin barrier without the use of preservatives or broad-spectrum antibacterial agents, to comprehensively support skin barrier health and healing. The composition may include a defined proportion of emollients, physiologically compatible acids, plant-based or non-harming anti-inflammatories, and optionally a vasodilator. As used herein, the term “emollient” refers to any substance or mixture of substances that functions to soften, smooth, lubricate, or moisturize the skin or enhance the sensory feel of a formulation. Emollients may be hydrophobic (lipophilic), such as oils, waxes, butters, fatty acids, fatty alcohols, silicones, esters, hydrocarbons, and related compounds, or hydrophilic, such as polyols (e.g., glycerin), low molecular weight glycols, and other water-attracting agents that may contribute to skin hydration. Hydrophilic emollients may include compounds such as glycerin, sorbitol, propylene glycol, and similar substances. The term “emollient” is intended to encompass both natural and synthetic materials, and may include single compounds or mixtures thereof. Unless otherwise specified, the term is not limited by chemical structure, polarity, or source and examples enumerated above are non-exhaustive.

In accordance with an aspect, there is provided a topical, anhydrous skin-barrier repair composition for application to human skin exhibiting irritation, inflammation, or barrier disruption, the composition having a 5-35 wt % of one or more emollients, a 1-35 wt % of at least one physiologically compatible acid, a 0.1-25 wt % of at least one naturally-sourced, or non-harming anti-inflammatory agent, 0-5 wt % of a vasodilator (optional), wherein each component is selected and proportioned so that the composition (i) moisturizes without causing further irritation, (ii) maintains or restores the skin's natural acidity, (iii) down-regulates cutaneous inflammation based on the severity of the inflammation, and (iv) doesn't hinder a healthy skin microbiome.

In a related aspect the total water content is below 0.5 wt %, thereby providing a waterless balm resistant to microbial spoilage without added preservatives.

In a related aspect the acid fraction is 5-30 wt %.

In a related aspect the anti-inflammatory agent is selected from calendula officinalis extract, oat kernel oil, or seabuckthorn oil or other non-harming anti-inflammatory.

In a related aspect the vasodilator is oil soluble.

In a related aspect, the use of a composition described herein is for the manufacture of a medicament for the treatment or prevention of atopic dermatitis, psoriasis, or other inflammatory skin conditions characterized by barrier dysfunction.

In a related aspect, the method of manufacturing the topical skin-barrier repair composition includes dissolving one or more physiologically compatible acids in one or more emollients, with one or more naturally-sourced or non-harming anti-inflammatory agents under controlled temperature, wherein the water content is below 0.5% by weight, with the physiologically compatible acid being at a percentage suitable for skin re-acidification.

In a related aspect, the composition comprises a triglyceride emollient, at least one physiologically compatible acid, at least one naturally-sourced or non-harming anti-inflammatory agent; and a wax or solidifying agent for rendering the composition self-supporting, wherein the at least one physiologically compatible acid is dissolved in the triglyceride emollient and wherein the composition is substantially free of water.

Further areas of applicability will become apparent from the description provided herein. The description and specific examples in this summary are intended for purposes of illustration only and are not intended to limit the scope of the present disclosure.

### DETAILED DESCRIPTION

In the following description, details are set forth to provide an understanding of the present disclosure.

The skin barrier, primarily composed of the stratum corneum, is a complex, multi-layered structure that serves as the body's first line of defense against environmental insults, pathogens, and water loss. This intricate barrier is maintained by a combination of physical, chemical, microbiome, and immunologic layers. Disruption of any of these layers can result in increased transepidermal water loss (TEWL), elevated skin pH, dysbiosis of the skin microbiome, and heightened inflammation, manifesting clinically as dryness, erythema, pruritus, and increased susceptibility to infection.

A critical aspect of skin barrier function often overlooked is the maintenance of an acidic surface pH, typically between 4.0 and 6.0. This acidity is essential for regulating key enzymes involved in lipid processing and barrier formation, supporting the growth of beneficial commensal flora while suppressing pathogenic microbes, promoting barrier repair and reducing inflammation, and optimizing the activity of proteases and other enzymes involved in desquamation and cornification. Disruption of the acid mantle, common when using alkaline skincare products or caused through environmental insults, impairs these processes, leading to barrier dysfunction and inflammation.

Current commercially available pH-balanced products often fail to address the importance of skin acidity. Many lack anti-inflammatory components, or include preservatives and broad-spectrum antibacterials that disrupt the skin microbiome. The challenge of formulating a preservative-free, acidic product that delivers a physiologically relevant pH upon application has not been adequately addressed in the art. There is a pressing need for topical formulations that can restore and maintain the skin's natural acidity, support a healthy microbiome, provide effective moisturization, reduce inflammation, and restore the skin barrier without the use of preservatives or broad-spectrum antibacterial agents.

It is important to note that, at present, the only practical method for creating a preservative-free skincare formulation is through the use of anhydrous (waterless) systems. The absence of water in such formulations inherently inhibits microbial growth, thereby obviating the need for traditional preservatives that may themselves cause irritation or disrupt the skin barrier. However, a significant technical limitation of anhydrous compositions is that conventional pH measurement is not feasible, as pH is a property of aqueous solutions and cannot be directly determined in the absence of water. As a result, the present disclosure is based on empirical testing of the formulation's effects on human skin, with particular attention to tolerability, skin re-acidification, and barrier repair, rather than on pH values measured in the product itself. This approach departs from standard practices that rely on pH adjustment and monitoring in aqueous systems and instead focuses on the real-world performance and safety of the anhydrous composition when applied to compromised skin.

Accordingly, the composition may provide a suitable option for individuals with skin barrier disruption, irritation, or inflammatory skin conditions such as atopic dermatitis, rosacea, or psoriasis.

The drawbacks of existing technology in the field of topical skin-barrier repair compositions may include for example: (1.) Most commercial barrier repair products fail to address the importance of maintaining the skin's natural acidic pH, which is crucial for optimal barrier function and overall skin health. (2.) Existing formulations lack effective anti-inflammatory components, or include preservatives and broad-spectrum antibacterial agents that disrupt the skin's natural microbiome. (3.) The challenge of formulating an anhydrous, acidic product that delivers a physiologically relevant pH upon application has not been adequately addressed in the art, limiting the effectiveness of current barrier repair solutions. (4.) Current topical treatments often lack the ability to be tailored for optimal efficacy and tolerability across a spectrum of skin conditions, particularly for individuals who are prone to severe or chronic skin disruptions.

Thus, many existing formulations focus on single mechanisms of action, failing to provide a comprehensive approach that simultaneously addresses multiple aspects of skin barrier restoration and maintenance, such as pH regulation, moisture retention, inflammation reduction, and microbiome support.

There is provided herein dermatological compositions and methods for supporting and repairing the skin barrier following disruption. A preservative-free, acidic balm that restores the skin's acid mantle, supports the microbiome, and calms inflammation, thereby preventing the progression of minor irritation to chronic or severe skin inflammation or restoring the skin after major irritation based on the ratio of the acids and anti-inflammatories used in the formulation will now be described in more details.

A topical, anhydrous skin-barrier repair composition is provided having at least the following illustrative composition formed for acidification, moisturization, anti-inflammatory action, microbiome support, and (optionally) vasodilating properties.

Acidification aspects may include a composition containing 1-35 wt % of at least one physiologically compatible acid, conferring an acid value sufficient to achieve acidification ideal for restoring healthy skin pH. This restores and maintains the skin's acid mantle, optimising enzymatic activity and microbial balance as well as promoting barrier repair and reducing inflammation.

Moisturisation aspects may include the inclusion of any combination of emollients which provide effective hydration and occlusion, reducing TEWL without causing further irritation.

Anti-Inflammatory aspects may include naturally-sourced, or non-harming, anti-inflammatory agents, (contrary to steroids, for example, that may thin the skin over time and hurt the skin barrier) that calm inflammation and support barrier recovery.

Microbiome support aspects may include a formulation that is acidic in nature and free from preservatives or broad-spectrum antibacterials, thereby helping to preserve the diversity and health of the skin microbiome.

Vasodilator aspects may be optionally included for accelerated healing.

In accordance with an illustrative embodiment, the composition may include following components (1) Emollient: 5-35 wt % (2) Physiologically Compatible Acid: 15-30 wt % (3) Naturally-Sourced or Non-Harming Anti-Inflammatory Agent: 1-30 wt % (4) Vasodilator (optional): 0-5 wt % (e.g., oil soluble vasodilator).

In a preferred embodiment, for initial barrier disruption, the ingredients may include approximately: 16% Caprylic/Capric Triglyceride, 11% Simmondsia chinensis (Jojoba) Seed Oil, 5% Squalane, and 11% Butyrospermum parkii (Shea) Butter, 20% Oleic Acid, 4% Hippophae rhamnoides (Seabuckthorn) Fruit Oil, 4% Avena sativa (Oat) Kernel Oil, 4% Calendula officinalis Flower Extract, 5% Euphorbia cerifera (Candelilla) Wax, 3% Hydrogenated Soybean Oil, 3% Glyceryl Behenate, 3% Cetyl Palmitate, 3% Trihydroxystearin, 2.5% Octyldodecyl Myristate, 1% Tocopherol, 1% Tetrahexyldecyl Ascorbate, 1% Hydrogenated Castor Oil/Sebacic Acid Copolymer, and 0.5% Vanillin. Optionally, up to 2% of an oil-soluble vasodilator can be included.

Another alternate, but preferred, embodiment for prolonged barrier disruption and heightened skin inflammation, may include approximately: 16% Caprylic/Capric Triglyceride, 11% Simmondsia chinensis (Jojoba) Seed Oil, 5% Squalane, 11% Butyrospermum parkii (Shea) Butter, 5% Oleic Acid, 8% Hippophae rhamnoides (Seabuckthorn) Fruit Oil, 8% Avena sativa (Oat) Kernel Oil, 9% Calendula officinalis Flower Extract, 5% Euphorbia cerifera (Candelilla) Wax, 3% Hydrogenated Soybean Oil, 3% Glyceryl Behenate, 3% Cetyl Palmitate, 3% Trihydroxystearin, 4.5% Octyldodecyl Myristate, 1% Tocopherol, 1% Tetrahexyldecyl Ascorbate, 1% Hydrogenated Castor Oil/Sebacic Acid Copolymer, 0.5% Vanillin, and optionally 2% of an oil-soluble vasodilator.

This composition is anhydrous, meaning it does not contain water, which helps maintain stability and prevent microbial growth. The formulation is free from preservatives and broad-spectrum antibacterial agents, instead relying on the acidic environment and natural components to support a healthy microbiome.

To achieve the desired pH and optimize efficacy, the composition is prepared in accordance with one possible illustrative example as follows:
1. The physiologically compatible acids are dissolved in a mixture of triglyceride emollients and non-polar emollients at elevated temperatures (60-80° C.).
2. The anti-inflammatories and vasodilator (if used) are added to the lipid phase and dispersed evenly.
3. Emollient waxes or solidifying agents are melted and incorporated into the lipid phase.
4. The resulting mixture is homogenized and cooled to room temperature.

Due to the inability to test pH in an anhydrous (water-free) formulation, the specific percentages described herein result from arduous testing.

The composition is applied topically to the skin in a thin layer, preferably after cleansing and drying the skin. It can be used as often as needed, depending on the severity of the skin condition, typically 1-3 times daily. The composition can be incorporated into a routine skin care regimen, including after bathing or showering, before makeup application, or as needed throughout the day.

The topical, preservative-free skin-barrier repair composition provides a comprehensive approach to restoring and maintaining the skin's natural acidity, supporting a healthy skin microbiome, and addressing various aspects of skin barrier function. The precise acidification, combined with the carefully selected and optimized components, makes this composition unique in the field of skin care and potentially addresses the limitations of existing barrier repair products.

The topical, preservative-free skin-barrier repair composition may have the following beneficial effects, including but not limited to:
1. The composition effectively aids in the re-acidification of skin after barrier disruption, which is crucial for optimal barrier function and overall skin health. This is achieved through the precise acidification of the formulation, accomplished by incorporating a controlled content of one or more physiologically compatible acids.
2. The composition supports a healthy and diverse microbiome by providing an environment that promotes the growth of beneficial commensal flora while suppressing pathogenic microbes. This is accomplished by maintaining an acidic environment and avoiding the use of preservatives or broad-spectrum antibacterial agents.
3. The composition provides effective moisturization and occlusion by incorporating emollients, helping to reduce transepidermal water loss (TEWL) without causing further irritation.
4. The composition offers anti-inflammatory action through the use of naturally-sourced or non-harming anti-inflammatory agents, allowing for effective calming of inflammation and support of barrier recovery. This approach avoids the potential side effects associated with long-term use of steroids (and other similar pharmaceutical applications), such as thinning of the skin.

The composition can be tailored for optimal efficacy and tolerability across a spectrum of skin conditions by calibrating the ratio of acids to anti-inflammatories according to the severity and type of skin barrier disruption. This personalized approach addresses the limitations of existing formulations that lack the ability to adapt to various skin conditions.

The present teachings may be effective for individuals with barrier-compromised skin, including those with atopic dermatitis, psoriasis, or general skin sensitivity.

The present teachings are also beneficial when used in combination with irritating skincare products like retinol, whereby using a variation of this combination with regular retinol use will prevent the skin irritation often associated with the use of retinol and similar actives.

The present teachings are applicable in the fields of dermatology, cosmetics, and personal care and provide for the prevention and treatment of skin barrier dysfunction, with advantages over existing products in terms of efficacy, safety, and microbiome preservation. The preservative-free, acidic barrier repair balm described addresses the critical role of skin acidity in barrier function, microbiome health, and inflammation control. The composition described is formulated to deliver consistent, effective results without the drawbacks of conventional barrier repair products or steroids. The present composition may be anhydrous (water-free) to avoid the use of preservatives. The present composition combines acidity with an anti-inflammatory to achieve the re-acidification of the skin and repair the skin barrier, while downregulating skin inflammation. The present composition uses acidity to help with skin re-acidification.

Clearly, changes may be made to what is described and illustrated herein without, however, departing from the scope defined in the accompanying claims. The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

What is claimed is:

1. A topical, preservative-free skin-barrier repair composition for application to human skin the composition comprising one or more physiologically compatible acids to aid in skin re-acidification.

2. The composition of claim 1, wherein the one or more physiologically compatible acids are dissolved in one or more emollients.

3. The composition of claim 1, wherein the composition is substantially water-free.

4. The composition of claim 1, wherein the total water content of the composition is below 0.5% by weight.

5. The composition of claim 1, further comprising one or more vasodilators, wherein the one or more vasodilators is employed in a total amount of about 0-5% by weight.

6. The composition of claim 1, wherein the one or more physiologically compatible acids is employed in a total amount of about 1-25% by weight.

7. The composition of claim 1, further comprising one or more anti-inflammatory agents, wherein the one or more anti-inflammatory agents is employed in a total amount of about 1-35% by weight.

8. The composition of claim 7, wherein the one or more anti-inflammatory agents is at least one of a naturally-sourced or non-harming anti-inflammatory agents.

9. The composition of claim 7, wherein the one or more anti-inflammatory agents is employed in a total amount of about 1-25% by weight.

10. The composition of claim 1, further comprising one or more emollients, wherein the one or more emollients is employed in a total amount of about 1-90% by weight.

11. The composition of claim 10, wherein the one or more emollients is employed in a total amount of about 1-80% by weight.

12. The composition of claim 11, wherein the one or more emollients is employed in a total amount of about 1-70% by weight.

13. The composition of claim 12, wherein the one or more emollients is employed in a total amount of about 1-60% by weight.

14. The composition of claim 13, wherein the one or more emollients is employed in a total amount of about 1-50% by weight.

15. The composition of claim 1, further comprising at least one anti-inflammatory agent, and one or more emollients having the one or more physiologically compatible acids incorporated through solvation, wherein each component is selected and proportioned so that the composition (i) moisturizes, (ii) maintains or helps to restore the skin's natural acidity, (iii) down-regulates cutaneous inflammation, and (iv) sustains a diverse skin microbiome.

16. The composition of claim 1, wherein the composition is used for the manufacture of a medicament for the treatment or prevention of atopic dermatitis, psoriasis, or other inflammatory skin conditions characterized by barrier dysfunction.

17. A method of manufacturing a topical skin-barrier repair composition for application to human skin, the method comprising
providing one or more physiologically compatible acids to aid in skin re-acidification;
providing one or more emollients;
dissolving the one or more physiologically compatible acids in the one or more emollients.

18. The method of manufacturing of claim 17, further comprising blending the one or more emollients with the one or more physiologically compatible acids, and with one or more naturally-sourced or non-harming anti-inflammatory agents under controlled temperature; and mixing the blended composition to ensure homogeneity.

19. The method of claim 18, further comprising adjusting the final composition to achieve a water content below 0.5% by weight, with the physiologically compatible acid being at a percentage suitable for skin re-acidification.

20. A topical, preservative free, anhydrous skin-barrier repair composition for application to human skin, the composition comprising:
a triglyceride emollient;
at least one physiologically compatible acid;
at least one naturally-sourced or non-harming anti-inflammatory agent; and
a wax or solidifying agent for rendering the composition self-supporting;
wherein the at least one physiologically compatible acid is dissolved in the triglyceride emollient;
wherein the composition is substantially free of water.
